(19) United States
(12) Patent Application Publication
(10) Pub. No.: US 2013/0289383 A1
(43) Pub. Date: Oct. 31, 2013

(54) Magnetic Relaxometry using Brownian Randomization, Neel Relaxation, or Combinations Thereof

(71) Applicants: Edward R. Flynn, Albuquerque, NM (US); Victor Gerald Grafe, Corrales, NM (US)

(72) Inventors: Edward R. Flynn, Albuquerque, NM (US); Victor Gerald Grafe, Corrales, NM (US)

(21) Appl. No.: 13/871,683

(22) Filed: Apr. 26, 2013

Related U.S. Application Data
(60) Provisional application No. 61/639,827, filed on Apr. 27, 2012.

(51) Int. Cl. A61B 5/05

(52) U.S. Cl. CPC A61B 5/05
USPC 600/409

(57) ABSTRACT

The present invention can provide a method of determining the communication of substances between a first region and a second region of a patient's body. An example method according to the present invention can comprise: (a) introducing into the first region a plurality of superparamagnetic nanoparticles, having properties such that they undergo Brownian motion that randomizes the orientation of the nanoparticles according to a predetermined characteristic time; (b) after a time sufficient to allow transport of nanoparticles from the first region to the second region, subjecting the second region to an applied magnetic field of sufficient strength to induce magnetization of individual nanoparticles, and having a substantially uniform direction throughout the second region; (c) measuring the magnetic field of the second region at a plurality of times after ceasing application of the magnetic field; (d) analyzing the measured magnetic field to detect signals that correspond to decay of the magnetic field due to randomization of the nanoparticles' orientation by Brownian motion; (e) determining the presence of nanoparticles in the second region from the signals detected in step (d).

### CROSSREFERENCE TO RELATED APPLICATIONS

This invention claims priority to U.S. application 61/639,827, filed Apr. 27, 2012, which is incorporated herein by reference.

### FIELD OF THE INVENTION

This invention relates to the in vivo detection and measurement of cells or substances using nanoparticles and magnetic relaxation measurements.

### BACKGROUND OF THE INVENTION

Early detection is of utmost importance in cancer detection for the most likely chance of survival. However, in most cancer situations, detection of an existing cancer must be augmented by determination if the cancer has spread. In the case of surgery, the first step is by removing sufficient tissue to not only remove the tumor but sufficient surrounding tissue to assure that the margins around the tumor contain no cells. To further ascertain that the cancer has not spread beyond the original source, it is important to identify the nearest lymph nodes, often referred to as the sentinel nodes, where the flow of lymphatic fluid would have carried metastatic cancer cells. The lymph nodes are normally removed (lymphadenectomy) and examined for the presence of cancer cells that might be emanating from the primary site and an indication that the cancer has metastasized requiring substantially more therapy. Removal of the lymph nodes is also done for further assurance of stopping the spread of cancer. This is done for many types of cancer where the lymph nodes can be identified, including breast, skin, ovarian, prostate, and others.

There are two primary existing methods for identifying the sentinel node or nodes where the lymph body fluids would be accumulating from the region of the cancer lesion. One of these is the injection of a blue dye into the tumor and then to examine extracted lymph nodes from nearby regions to see if the dye is present. This method requires prior knowledge of the lymph node and surgical extraction. As such, the surgical intervention may be proven unnecessary if the result is negative or if the sentinel node was misidentified. Removal of lymph nodes always has unfortunate consequences including buildup of fluids at the surgical site, swelling of nearby limbs, infection, numbness and local pain.

Another method, that does not require prior knowledge of the node location and resulting surgical removal, is to inject a radioactive isotope, commonly technetium sulfur colloid, into the tumor and localize the flow of the isotope by measuring the emitted radiation with detectors. Detectors may include small hand-held sensors or more sophisticated sensors such as Single-Photon emission computed tomography (SPECT) or Positron-Emission tomography (PET). This method involves radiation exposure to the subject and must be used sparingly. The results of this localization is to identify the sentinel node although it does not measure the presence of cancer cells in the lymph node and surgical removal of the node is required to determine this.

There is a need for methods and apparatuses that can identify sentinel lymph nodes, and trace other flow patterns in the body, that do not require prior knowledge of the flow, do not require harmful radiation, and can reduce the need for surgery.

### BRIEF DESCRIPTION OF THE DRAWINGS

The accompanying drawings, which are incorporated in and form part of the specification, illustrate the present invention and, together with the description, describe the invention. In the drawings, like elements are referred to by like numbers.

FIG. 1 is a schematic illustration of an example preparation of tissue of a subject for measurement according to the present invention.

FIG. 2A, FIG. 2B, FIG. 2C, and FIG. 2D provide a schematic illustration of an example measurement in accord with the present invention.

FIG. 3 is a schematic illustration of measurements from the process described in connection with FIG. 2.

FIG. 4 is a schematic illustration of an apparatus suitable for use in the present invention.

FIG. 5 is a schematic illustration of an exemplary apparatus using superconducting quantum interference device (SQUID) magnetic sensors.

FIG. 6 is a schematic illustration of an exemplary SQUID sensor apparatus that can be used for human cancer examinations.

FIG. 7 is a schematic illustration and photo of an atomic magnetometer for weak field measurements.

FIG. 8 is schematic illustration of a magnetic nanoparticle with biocompatible coating and attached antibodies for targeting specific cells.

FIG. 9 is a depiction of the number of Her2 sites per cell calculated by comparison to a range of microspheres with known binding capacities.

FIG. 10 is an illustration of magnetic moments of two breast cancer cell lines, MCF7/HER218 and MDA-MB-231 measured as function of time after incubating with HER2/neu antibodies and nanoparticles.

FIG. 11 is an illustration of the magnetic moments of cell samples measured as function of number of cells by pipetting cells down by factors of two.

FIG. 12 is an illustration of a phantom with inserted vials of MCF7 Cells, left 2E+06, right=1E+06 cells.

FIG. 13 is a photo of a nude mouse under a SQUID system.

FIG. 14 contains position confidence plots obtained from mouse tumors.

FIG. 15 is an illustration of the magnetic contour lines observed for 35 different measurement sites

FIG. 16 is an illustration of the time course of the measurements for the two mice and both tumors of each mouse.

FIG. 17 is an illustration of the results of these measurements and show very good agreement with the in-vivo measurements on the live mouse.

FIG. 18 is an illustration of the 2-dimensional 95% confidence limit for the locations of the two tumors superimposed on the actual tumors of the mouse.

FIG. 19 is a photo of the histology of tumors after extraction.

FIG. 20 is an illustration of an ovarian cancer showing the growth of the tumor on the ovary.

FIG. 21 is photograph of a full-size ovarian phantom placed under a SQUID sensor apparatus at a distance that would be typical of a patient subject.

FIG. 22 is an illustration of the results of sensitivity studies for live ovarian cells inserted into the phantom shown in FIG. 21.

FIG. 23A and FIG. 23B provide an illustration of confirmation of antibody sites for these cells using flow cytometry.

FIG. 24 is an illustration of magnetic moments from magnetic nanoparticles (from Ocean Nanotech) attached to ovarian human cancer tumors in the live mouse.

FIG. 25 is a photograph of a mouse used to verify that the SQUID sensor method works in-vivo along with magnetic contour fields from the mouse.

FIG. 26 is a graph of a measurement of the magnetic moment in a SQUID sensor system as a function of time for incubation of attaching magnetic nanoparticles to lymphoma cell lines.

FIG. 27 is an illustration of the results of the flow cytometric measurements of RS cells from the lymphatic system in determining the number of sites available for nanoparticles and detection by the SQUID sensors.

FIG. 28 is a histology slice from a patient with Hodgkin's Disease.

FIG. 29 is a schematic illustration of results obtained from measuring, with an embodiment of the present invention, prostate cancer cells with PSMA targeted nanoparticles.

FIG. 30 is an illustration of magnetic field relaxation times for Neel and Brownian mechanisms.

FIG. 31 is a schematic illustration of the application of the present invention to detection of sentinel nodes.

### DISCLOSURE OF INVENTION

Embodiments of the present invention use superparamagnetic relaxometry (SPMR) combined with specialized magnetic nanoparticles. In some embodiments, the nanoparticles are injected into the tumor and superparamagnetic magnetic relaxation (SPMR) magnetic sensors are used to determine the path of the lymph flow and the location of the lymph nodes where the particles are accumulating. In one application of this method, the magnetic nanoparticles have a sufficiently large magnetic core and overall (hydrodynamic) diameter that when magnetized by a small magnetic field the magnetization would remain for several minutes, with the net magnetic moment decaying slowly by Brownian motion of the particles, and be measured by the SPMR sensors over time to determine the flow and ultimate accumulation node(s). This method requires no radiation, expensive radiation sensors, or nuclear medicine expertise. The resulting sentinel node determined by this method can then be extracted and examined without concern for radioactivity.

In many cases, the cancer will have well determined antibodies and the SPMR method can then take advantage of this to use smaller superparamagnetic nanoparticles to inject into the tumor contemporaneous with, instead of, or following the Brownian motion particles and examine the magnetic fields emanating from the sentinel node determined by the larger particles. In this case, magnetic fields emitted from the node indicate the presence of cancer in the node as the SPMR method is sensitive only to cancer cells that have been targeted by the antibody labeled nanoparticles. The SPMR technique has been shown to be extremely sensitive and when no emitted fields are sensed from the node using the antibody labeled nanoparticles, the determination of no cancer present is commensurate with a pathological evaluation of the node given the statistical uncertainties associated with such scans. SPMR methods and apparatuses are described in US application 13870925, filed 26 Apr. 2013, which is incorporated herein by reference.

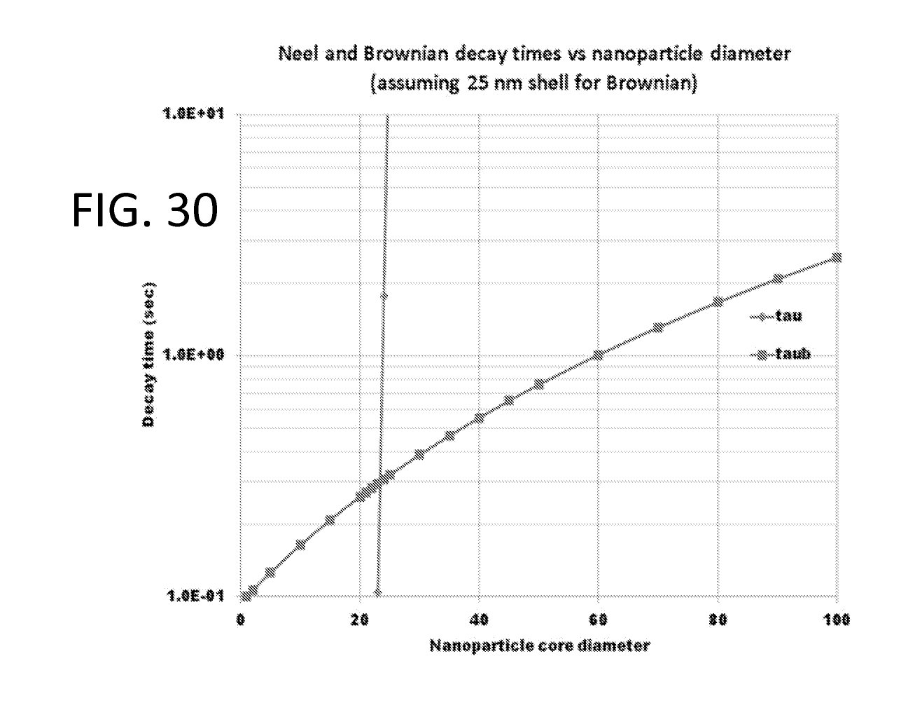

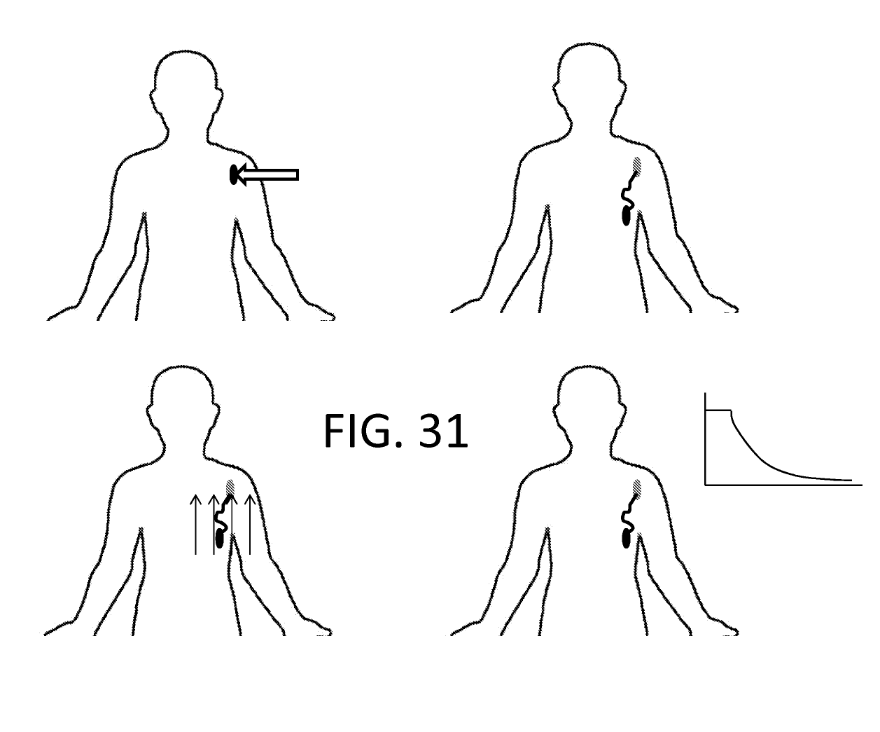

FIG. 30 illustrates the decay times for magnetic relaxometry of iron oxide nanoparticles of varying diameters. In the figure, “tau” refers to Neel time and “taub” refers to Brownian time. The Neel varies with core diameter as the exponential of the volume of the particle so is almost a straight vertical line in the figure. The Brownian varies as the volume. For this calculation, a thick shell around the core of 25 nm thickness was assumed to make the particle size commensurate with a sufficient slow time for measurement by SPMR. The viscosity of the medium was assumed to be 2. A careful selection of core size can allow use of the Neel decay time as well with core sizes somewhat larger than described elsewhere as used to select binding to cells vs. non-binding. As can be seen from the figure, the decay times can be tailored according to the size of the particles. The particle size and decay time for the magnetic relaxometry determination can be chosen to provide the desired measurement time. If the Brownian particle decay time is similar to that of a bound Neel particle, then the same magnetization and analysis methods can be used for both. If the particles are chosen such that the decay times are different, then the particles can be measured at the same time, and the two different decay times used to distinguish the signal from Brownian particles from that of Neel particles, allowing the flow paths to be determined in the same process as detection of the cancer or other target of the Neel particles.

The use of SPMR to determine the sentinel nodes associated with nearby cancer lesions offers significant advantages over other methods. First, it can be used to localize the sentinel node without the need to remove nodes first for examination that may prove false, without the need for radioactive tracers and without examination being contaminated with radioactivity. Secondly, when appropriate antibodies for the specific cancer are known, the method can be used to identify if the cancer has spread to the node without the necessity of surgical removal thus saving the patient considerable later discomfort.

The methods and apparatuses described herein can also be used to trace distribution paths in a body without radiation such as is required for PET scans. Nanoparticles suitable for measurement by detecting magnetic field decay due to Brownian motion (for convenience, called “Brownian particles” herein) can be introduced, and their position determined from magnetic relaxometry as described in connection with lymph nodes. Their distribution over time can be used to determine the route and rate of fluid communication followed by the particles, and can be used to detect concentrations that can indicate blockages of the communication system or other concentrations of particles. As an example, blockage of a lymph node can be determined by concentration of Brownian nanoparticles in the lymph node, and can indicate a lymph drainage abnormality such as can be caused by cancer present in the lymph node. Determinations of the flow of the Brownian particles over time can allow determination of flow rates in the communication network (e.g., lymph system). Brownian nanoparticles can also be attached to other substances such as pharmaceuticals, and the distribution of the other substance in the subject can be determined in vivo, and without surgery (or dissection in the case of animal models) by using magnetic relaxometry methods and apparatuses in accord with the present invention. As an example, the effectiveness of delivery of a pharmaceutical to a site can be determined noninvasively using the present invention.

Magnetic relaxometry methods and apparatuses suitable for use with the present invention are described in the following pages.

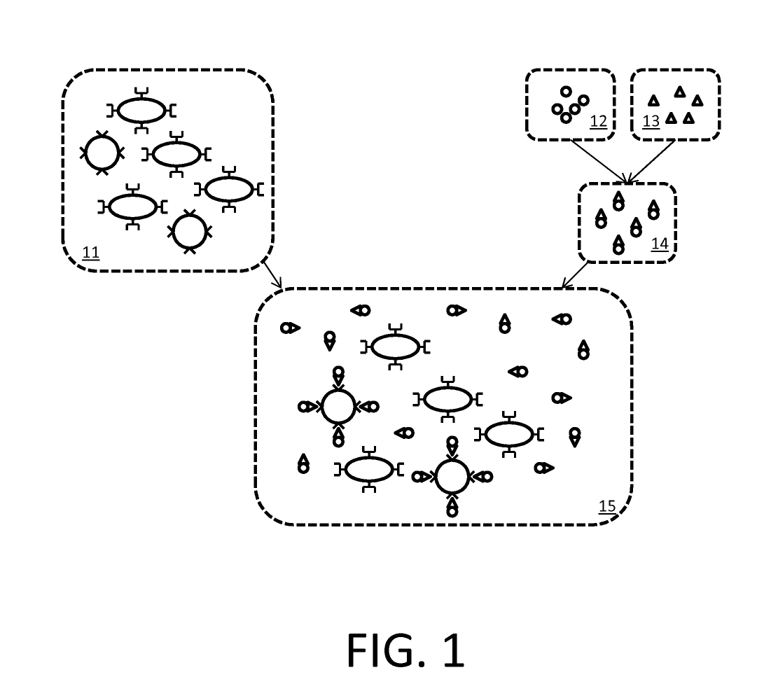

A simplified example of magnetic relaxation measurement using nanoparticles targeted to specific substances is first described. This targeting allows the discrimination between Neel relaxation and Brownian relaxation, since the particles that have bound to the targeted substance will have Brownian relaxation times much longer than would be expected from the particle size alone, since the particles' Brownian motion is hindered by their attachment to the relatively large substance (e.g., a cell). FIG. 1 is a schematic illustration of an example preparation of a sample for measurement according to the present invention. The illustrations in the figure are highly simplified and intended for ease of explanation only, and are not intended to represent the actual shapes, sizes, proportions, or complexities of the actual materials involved. A portion of the tissue 11, e.g., an organ to be investigated, or a known or suspected tumor site, or a cell culture, or a sample removed from a body, comprises some cells of the type of interest (shown in the figure as circles with “V” shaped structures around the periphery) and some cells of other types (shown in the figure as ovals with rectangular structures around the periphery). A plurality of magnetic nanoparticles 12 is provided, shown in the figure as small circles. A plurality of targeting molecules 13 is also provided, shown in the figure as small triangles. The nanoparticles and targeting molecules are combined (or conjugated), forming targeted nanoparticles 14.

The targeted nanoparticles can then be introduced to the tissue 15. Cells of the type of interest have binding sites or other affinities for the targeting molecule, illustrated in the figure by “V” shaped structures around the periphery of such cells. The targeting molecules attach to the cells of the type of interest, illustrated in the figure by the triangular targeting molecules situated within the “V” shaped structures. Generally, each cell will have a large number of such binding or affinity sites. Cells of other types do not have such binding sites or affinities, illustrated in the figure by ovals with no targeted nanoparticles attached. Targeted nanoparticles that do not bind to cells are left free in the prepared sample, illustrated in the figure by small circles with attached triangles that are not connected with any specific cell.

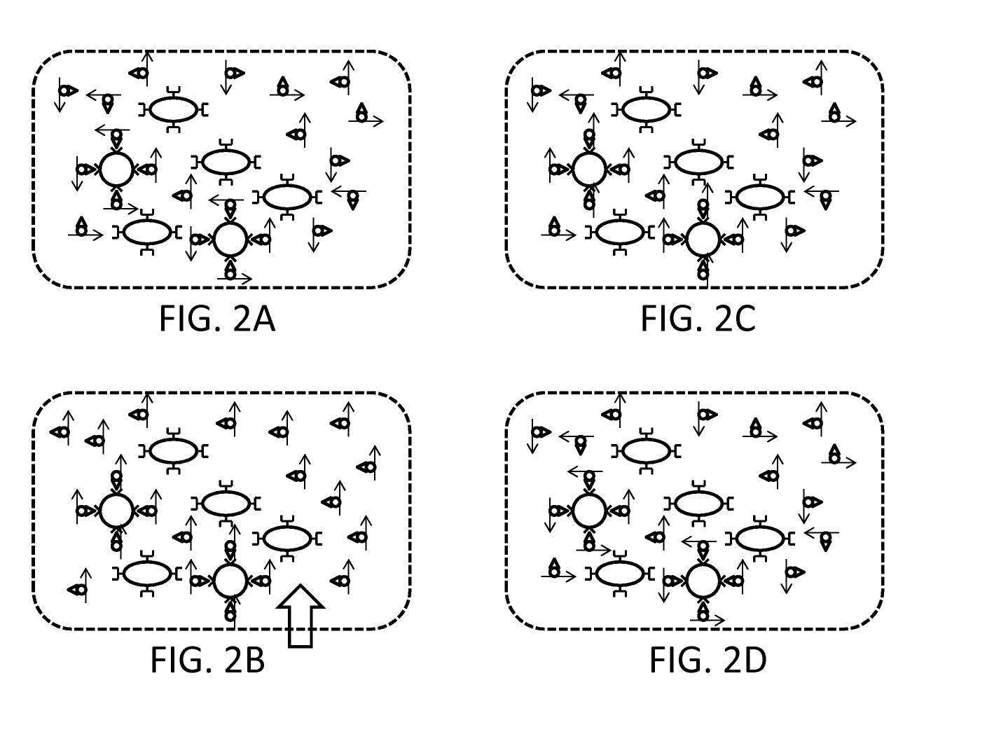

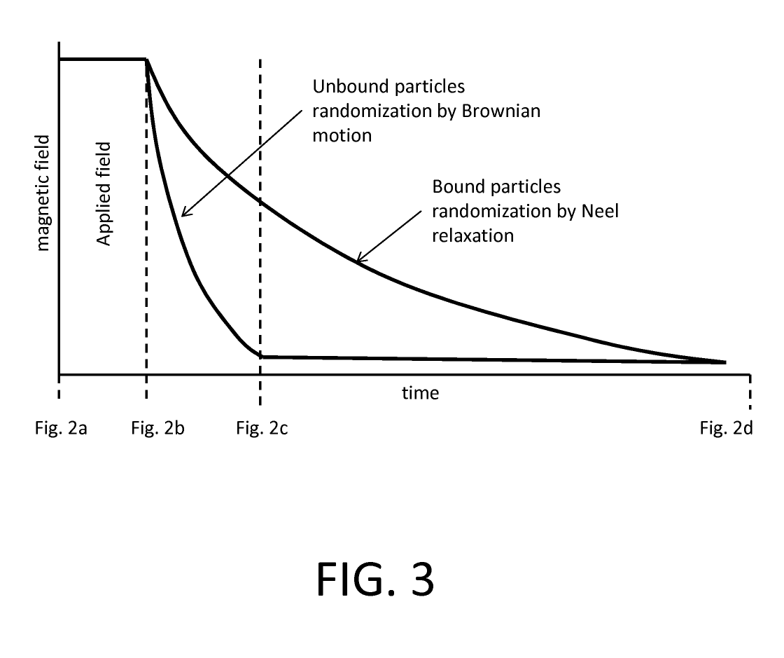

FIG. 2A, FIG. 2B, FIG. 2C, and FIG. 2D provide a schematic illustration of an example measurement in accord with the present invention. In FIG. 2A, the sample is as in FIG. 1, with the addition of arrows near each nanoparticle. The arrows are representative of the magnetization of each nanoparticle, and indicate that the magnetization of the nanoparticles in the tissue is random (in the figure, the arrows are shown in one of four directions for ease of illustration only; in practice the magnetization can have any direction).

In FIG. 2B, an external magnetic field (represented by the outlined arrow at the lower right of the figure) is applied. The magnetization of the nanoparticles in response to the applied magnetic field is now uniform, represented in the figure by all the magnetization arrows pointing in the same direction.

FIG. 2C illustrates the tissue a short time after the magnetic field is removed. The nanoparticles not bound to cells are free to move by Brownian motion, and their magnetization returns to random over a time interval defined by the Brownian motion characteristic of the unbound particles, represented in the figure by the magnetization arrows of the unbound nanoparticles pointing in various directions. The nanoparticles bound to cells, however, are inhibited from such physical motion and hence their magnetization remains substantially the same as when in the presence of the applied magnetic field.

FIG. 2D illustrates the prepared sample a longer time after removal of the applied magnetic field. The magnetization of the bound nanoparticles has by now also returned to random, by Neel relaxation of the magnetization of the particles.

FIG. 3 is a schematic illustration of measurements from the process described in connection with FIG. 2. Magnetic field is shown as a function of time in a simplified presentation for ease of illustration; in actual practice the units, scales, and shapes of the signals can be different and more complex. At the beginning of the process, corresponding to the state of FIG. 2A, the nanoparticle magnetization is random and the external magnetic field is applied. After that time, the magnetization of the nanoparticles is uniform, corresponding to the state of FIG. 2B.

The magnetic field contribution of the particles not bound to cells decays by Brownian motion during the transition from the state of FIG. 2B to that of FIG. 2C. Analyzing the measured magnetic field for a component that decays according to the curve expected for Brownian motion can allow the quantification and localization of those particles not bound to cells. In a tracing application, for example, there may be no targeted particles and all of the signal can be from unbound particles. The magnitude of the magnetization corresponding to Brownian decay, as a function of location in the sample, can be used to determine the number of particles that are present at each location in the sample.

The magnetic field contribution of the particles bound to cells decays by Neel relaxation during the transition from the state of FIG. 2C to that of FIG. 2D. The bound nanoparticles transition from uniform to random magnetization by Neel relaxation, corresponding to the state of FIG. 2D. The characteristics of the measurement magnetization from the state of FIG. 2C to that of FIG. 2D are related to the number of bound nanoparticles in the sample, and hence to the number of cells of the type of interest in the sample.

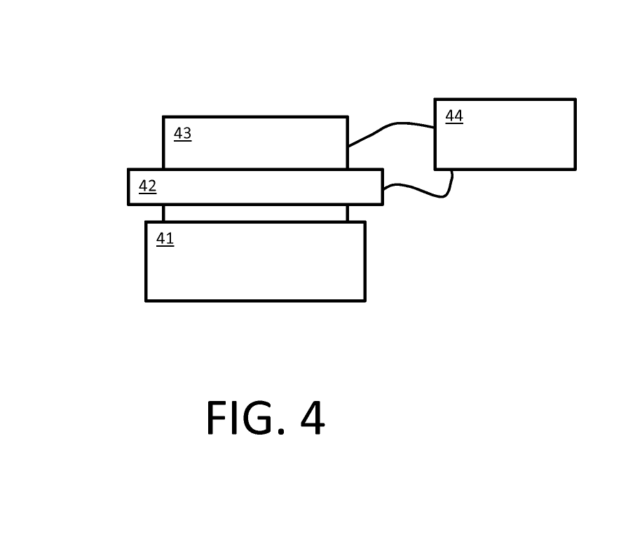

FIG. 4 is a schematic illustration of an apparatus suitable for use in the present invention. A sample stage 41 is configured to dispose the sample in an effective relationship to the rest of the apparatus. A magnetizing system 42, for example Helmholtz coils, mounts relative to the sample stage so that the magnetizing system can apply a magnetic field to the sample. A magnetic sensor system 43 mounts relative to the subject sample so that it can sense the small magnetic fields associated with the magnetized nanoparticles. The system is controlled and the sensor data analyzed by a control and analysis system 44; for example by a computer with appropriate programming.

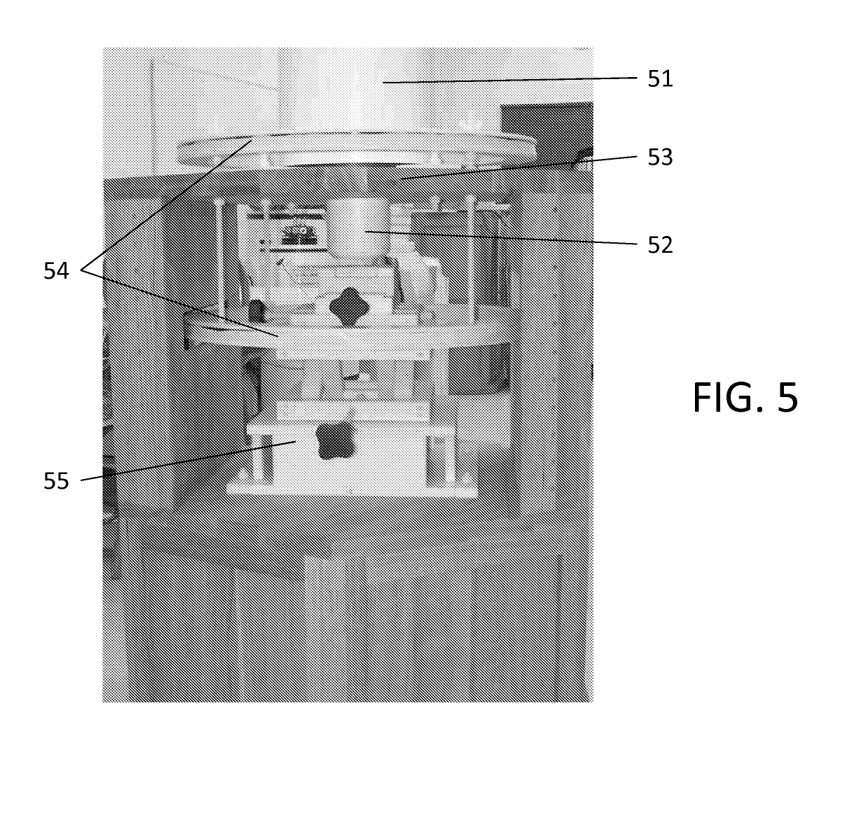

FIG. 5 is a schematic illustration of an exemplary apparatus using superconducting quantum interference device (SQUID) magnetic sensors. A liquid helium reservoir dewar 51 at the top of the picture maintains the temperature of the SQUID sensors. SQUID 2nd-order axial gradiometers are contained in a white snout 52 protruding through a support frame 53. There are seven gradiometers with a baseline of 4 cm contained within this exemplary snout; one in the center and 6 in a circle of 2.0 cm radius. Each gradiometer is inductively coupled to a low temperature SQUID. Two circular coils 54 form a Helmholtz pair that can provide a magnetizing pulsed field for the nanoparticles. The uniform field produced by these coils can be varied but typically is 40 to 50 Gauss and the pulse length is typically 300-800 msec. In this example, a wooden frame supports the SQUID and the measurement platform as well as the magnetizing coils. The non-magnetic support system comprises a 3-dimensional stage 55 that can be constructed with no metal components, e.g., of plastic. The upper two black knobs control the x-y stage movements over a +1-10 cm range and the lower knob is used to raise and lower the measurement stage over a 20 cm range. A sample holder can be inserted onto the stage that can contain cultures of live cancer cells, phantoms containing vials of live cancer cells and live subjects such as mice or other small animals.

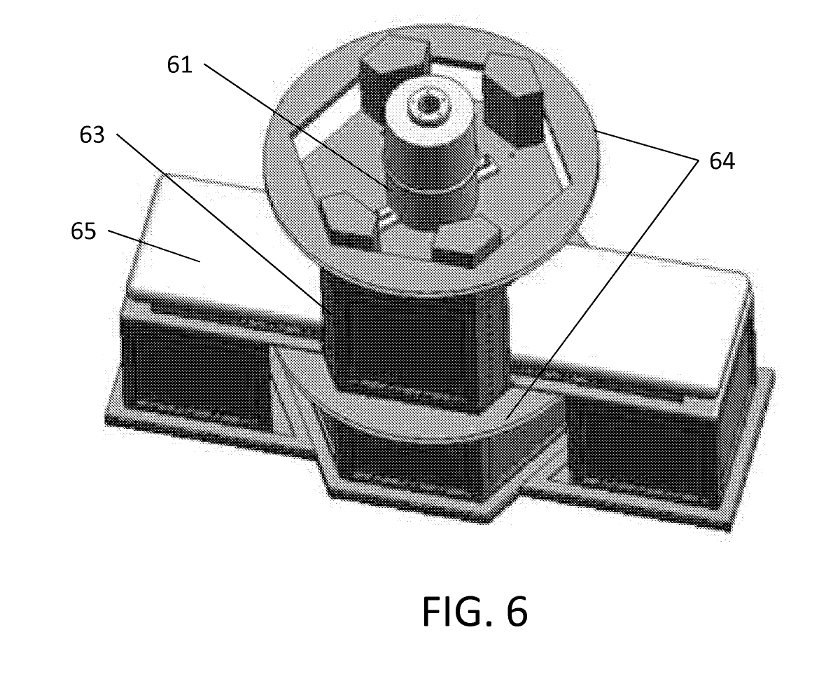

FIG. 6 is a schematic illustration of an exemplary SQUID sensor apparatus that can be used for human cancer examinations. A wooden structure 63 can be similar to the support frame shown in FIG. 5. The measurement stage can be replaced by a bed 65 for patient placement. Two larger Helmholtz coils 64 comprise the wooden circular forms above and below the bed. These larger coils can be used to generate a uniform pulse field and magnetize the magnetic nanoparticles that have been injected into the patient. The currents can be modified, e.g., increased, from those used in the apparatus shown in FIG. 5 to again produce fields in the range of 40 to 50 Gauss. Similar to the apparatus shown in FIG. 5, a SQUID dewar 61 with an array of magnetic gradiometers can be used to measure the residual magnetic field change produced by the magnetized nanoparticles.

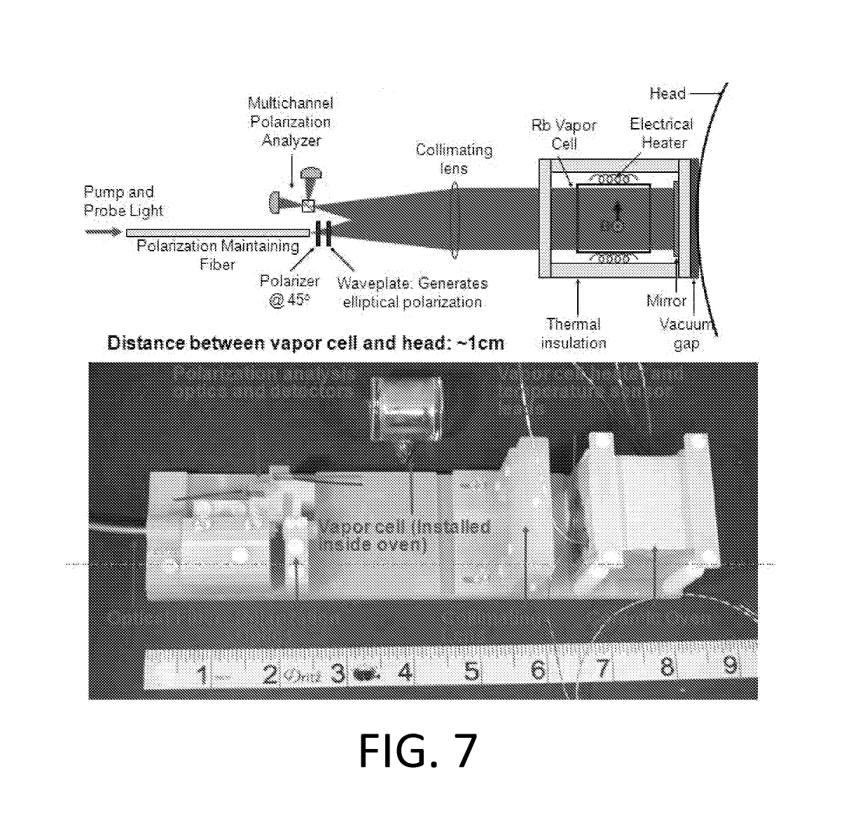

FIG. 7 is a schematic and photo of an atomic magnetometer suitable for use with some embodiments of the present invention. This device is miniaturized by using microchip fabrication methods and multiple units can be placed side-by-side to form an array of sensors. The operation of the magnetometer is through application of a laser light beam applied through an optical fiber. This beam pumps the heated Rb gas in the vapor cell into specific atomic states. The beam is first elliptically polarized and collimated into the vapor cell. A mirror reflects this beam back through the cell and lens into a polarization analyzer. A magnetic field applied perpendicular to the length of the magnetometer changes the index of refraction of the gas in the cell, changing the polarization of the light through the cell. The change in polarization yields the magnitude of the applied magnetic field. The pumping laser supplies multiple fiber optic cables and is thus used for multiple magnetometers. An array of these magnetometers for relaxometry measurements can comprise 7 vapor cells placed with one in the center surrounded by 6 more. The applied field from the magnetizing coils is perpendicular to the arrangement shown in FIG. 5 in order to induce the maximum observable magnetic moments into the nanoparticles. The photo at the bottom of FIG. 7 shows an exemplary physical arrangement and size of the atomic magnetometer for application with the present invention. The sensitivity of the device shown is 0.16 fT/VHz, compared to sensitivity of an exemplary SQUID system as shown in FIG. 5 of 1.0 pT/VHz (1000 fT/VHz). Atomic magnetometers require no cryogenic coolant which can make them desirable for clinical applications where such coolants, in particular liquid helium, are not always readily obtainable.

Example Application to Detection of Breast Cancer.

For breast cancer, the current method of choice for screening and detection is mammography. While mammography has led to a significant improvement in our ability to detect breast cancer earlier, it still suffers from the inability to distinguish between benign and malignant lesions, difficulty in detecting tumors in dense and scarred breast tissue, and fails to detect 10-30% of breast cancers. The use of magnetic nanoparticles conjugated to tumor-specific reagents combined with detection of these particles through measurement of their relaxing fields represents a promising new technology that has the potential to improve our ability to detect tumors earlier. Furthermore, detection of targeted magnetic nanoparticles using weak field sensors is fast and is can be more sensitive than MRI detection because only particles bound to their target cells are detected. This example application uses Neel relaxation to provide a measurement specific to breast cancer cells; the magnetic signal from Brownian decay of unbound particles in not used in this example.

We have developed conjugated magnetic nanoparticles targeted to breast cancer cells that express the HER2 antigen, which is overexpressed on ˜30% of human breast cancers. We have characterized the nanoparticles for their magnetic properties and selected those of optimal size and magnetic moment per mg of Fe. A number of different cell lines that have specificity to HER2 have been studied to determine their site density and sensitivity of the sensor system for detection. A SCID mouse model was explored using tumors grown from human cell lines, imaging the mouse under the sensor system followed by confirming histology studies. These results indicate the validity of the magnetic sensor approach for sensitive detection of breast cancer.

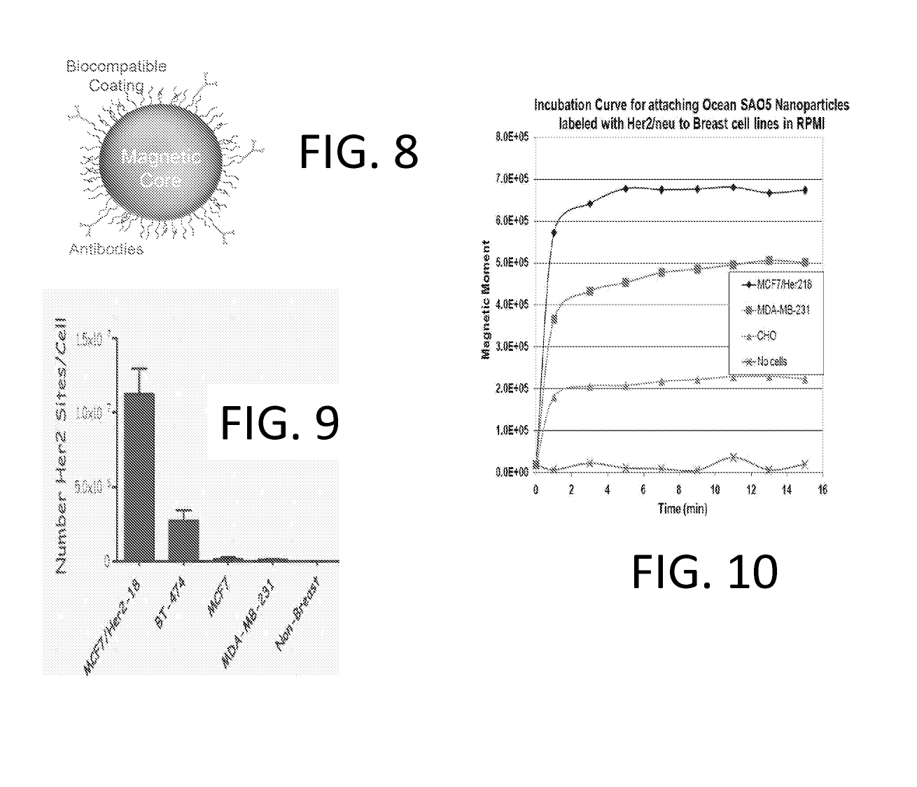

FIG. 8 is schematic illustration of a magnetic nanoparticle with biocompatible coating and attached antibodies for targeting specific cells. In a demonstration of an example embodiment of the present invention, we used HER2 Antibodies (Ab) that are specific to 30-40% of breast cancers. The nanoparticles had coatings containing Carboxyl groups and a Sulfo-NHS method is used to conjugate the nanoparticles to the antibodies. Flow cytometry performed for breast cancer cell lines MCF7, MCF7/Her2-18 (MCF7 clone stably transfected with Her2), BT474, and MDA-MB-231. Number of Her2 binding sites determined by flow cytometry, Anti-Her2 antibodies conjugated to the fluorescent probe FITC. FIG. 9 is a depiction of the number of Her2 sites per cell calculated by comparison to a range of microspheres with known binding capacities. MCF7 cells engineered to overexpress Her2-18 have 11×10⁶Her2 binding sites/cell, BT-474 have 2.8×10⁶, MCF7 0.18×10⁶, MDA-MB-231 0.11×10⁶ Non-breast cell lines have <4000 Her2 binding sites/cell.

FIG. 10 is a graph of a measurement of the magnetic moment in the SQUID sensor system as a function of time for incubation of attaching magnetic nanoparticles (from Ocean Nanotech) to breast cancer cell lines. The magnetic nanoparticles were coated with a carboxyl biocompatible coating and were then conjugated to the Her2/neu antibody. This antibody is specific to approximately 30% of breast cancer cells in humans. The labeled magnetic nanoparticles were inserted into vials containing live cancer cells and the magnetic moments of the vial measured at various times ranging from one minute to 16 minutes. The zero time point is the magnetic moment of the vial of nanoparticles before adding to the cells. The lack of magnetic moment for the unmixed particles is a demonstration that unbound particles give no magnetic signal with this SQUID imaging method. Upon mixing with the cells, the magnetic moments increase rapidly and saturate indicating that the cells have collected on their surfaces the maximum number of nanoparticles possible in one to two minutes. The top curve is for the breast cancer cell line, MCF7/Her218 that is known to be very specific for the Her2/neu antibody and the large magnitude of the magnetic signal verifies this. The breast cancer cell line, MDA-MB-231, is also positive for Her2/neu but with much fewer sites for the antibody targeted nanoparticles to attach to. The smaller magnitudes are also indicative of this trend. The CHO cell line is non-specific to Her2/neu and gives substantially smaller magnetic moments after incubation. The presence of a magnetic moment is indicative of some phagocytosis of these cells where the nanoparticles enter the cells. The curve for no cells is for the vials containing nanoparticles only and shows that the particles alone continue to give no signal and thus there is no agglomeration occurring of the particles. These results demonstrate the specificity of the antibody for the target cancer cells and verify that only bound particles give magnetic moments. This result is not true for other methods such as MRI which sees all particles, bound or unbound.

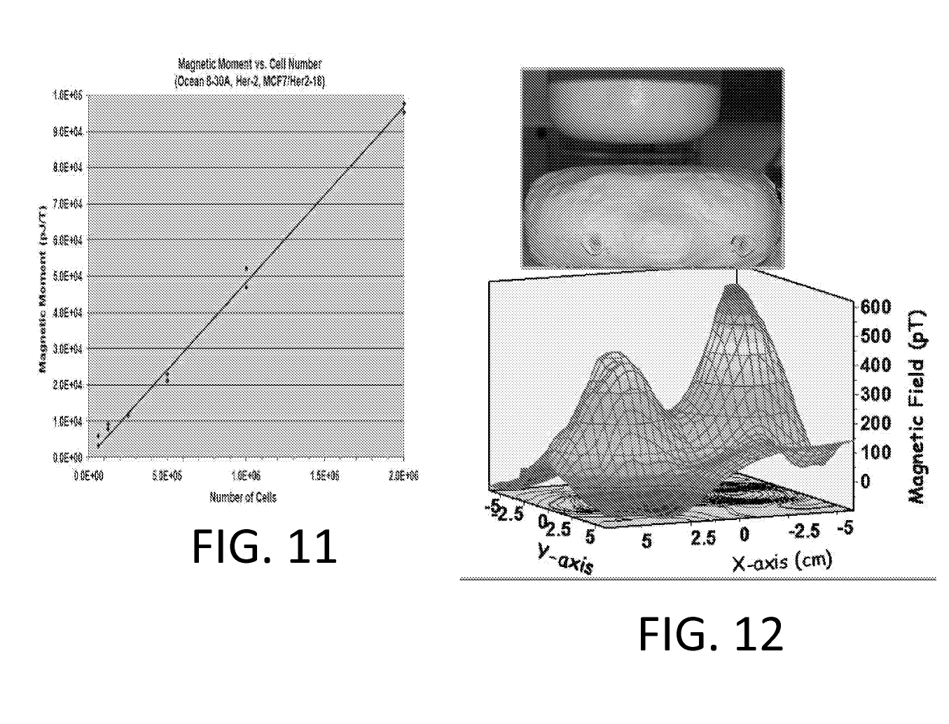

FIG. 11 is an illustration of the magnetic moments of cell samples measured as function of number of cells by pipetting cells down by factors of two. The demonstrated sensitivity is 100,000 cells for MCF7 cells and Ocean nanoparticles, for cells 3.5 cm from the sensor. There are 2.5×10⁶ np/cell. Linearity demonstrates magnetic moment yields # of cells; MRI contrast is not a linear function of cell number. A typical mammogram requires 10 million cells.

A breast phantom was constructed using a standard mammogram calibration phantom as a model. The phantom was constructed out of clay, non-metallic material is transparent to these fields. Vials containing live cells were inserted into the phantom. FIG. 12 is an illustration of a phantom with inserted vials of MCF7 Cells, left 2×10⁶, right=1×10⁶ cells. Cells conjugated to HER2 Ab, the np from Ocean Nanotech. Fields mapped at five 7-channel SQUID positions=35 sites. 3-D contour maps represent the field distributions. Locations and moment magnitudes obtained from inverse problem. Moments determine the number of cells in vials from cell data shown above.

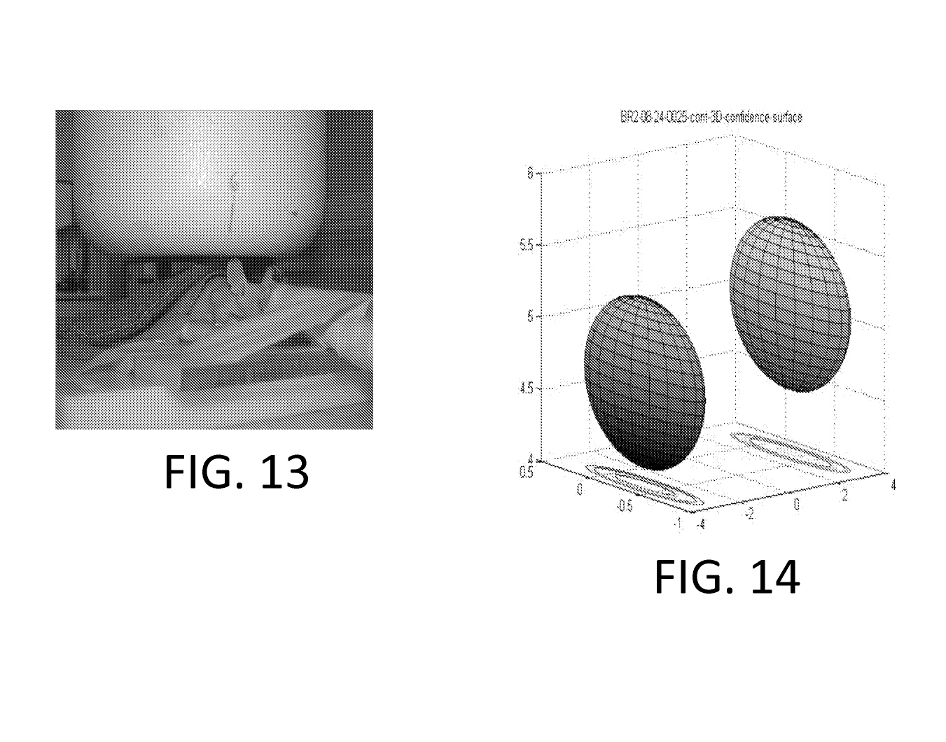

A mouse model of breast cancer was developed appropriate for SQUID sensor measurements. SCID nude mice were used with xenograft human breast cancer cell lines. FIG. 13 is a photo of a nude mouse under a SQUID system. To study in-vivo processes by the SQUID technique, a mouse was injected with human MCF7 cells two weeks previously in two places. These cells then produced human tumors on the flanks of the mouse; one such tumor is visible behind the right ear of the mouse. The mouse was anesthetized through the tube over its mouth. Labeled magnetic nanoparticles were injected into the mouse at this stage either by tail, inter-peritoneal, or inter-tumoral injections. Subsequent to injections, the mouse was placed under the sensor system as shown and a magnetizing pulse was applied and the resulting magnetic moments of the injected particles were measured. As in the case of the live cancer cells, no moments were observed unless the particles had attached to cells within the tumors. In some cases both tumors were MCF7 type cells and in other cases, two different cell lines were used to develop the tumors in the mice. The mouse resided on the stage shown in FIG. 5 and could be moved to several positions under the sensor system to obtain more spatial information. Measurements were made as a function of time to determine how fast the particles were taken up from the blood stream and how fast phagocytosis occurred with the particles ending up in the liver. The mouse was typically placed at five stage positions under the 7-channel SQUID system to obtain 35 spatial locations. The magnetic fields at all positions were then used in a special code to solve the electromagnetic inverse problem using the Levenberg-Marquardt theorem to determine the location of all sources of magnetic particles in the mouse. This information was then compared to the known geometry of the mouse from photographs to determine the accuracy and sensitivity for locating breast cancer tumors in living animals. FIG. 14 contains position confidence plots obtained from mouse tumors. Left sphere is from left tumor that is ˜2× right tumor in magnetic moment (see below). Positions calculated by two dipole least squares method to extract magnetic moments and positions. Moments determine number of labeled cells in tumors.

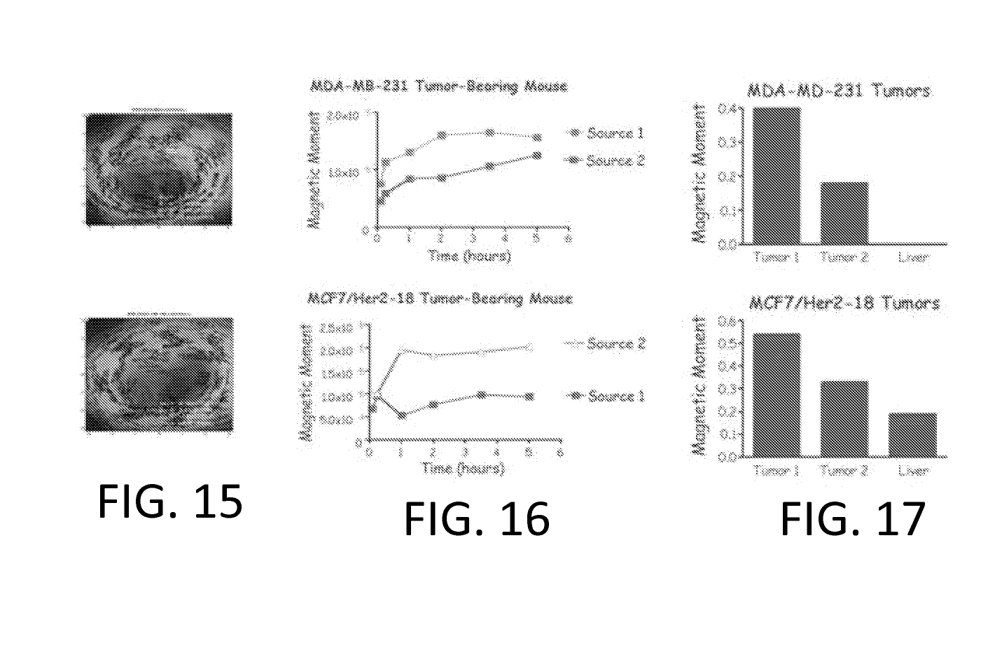

The SQUID system results for in-vivo measurements on living animals are shown in FIGS. 16, 17, 18 for two different tumor bearing animals. Each mouse had two tumors but of different cell types. Different amounts of nanoparticles were absorbed by each of the two tumors. The mouse with MCF7 cells showed higher magnetic moments than the mouse with MDA-MB-231 tumors as expected due to the higher number of specific sites for HER2/neu antibodies on the former. FIG. 15 is an illustration of the magnetic contour lines observed for 35 different measurement sites as described in FIG. 13. Analysis of these magnetic fields yielded the spatial positions of the tumors that agreed with the measured values of these positions; the SQUID results giving higher precision than the physical measurements of approximately 3 mm. FIG. 16 is an illustration of the time course of the measurements for the two mice and both tumors of each mouse. The uptake of the particles occurred rapidly with the signal near maximum obtained in the first hour. The nanoparticles remain in the tumors for at least 5 hours, the length of the experiments. Subsequent to these measurements, the mice were euthanized and the tumors and other organs removed and placed under the sensor system to determine how much of the nanoparticle injections were in the tumors. The plots in FIG. 17 are illustrations of the results of these measurements and show very good agreement with the in-vivo measurements on the live mouse. In the lower left figure, a magnetic moment was observed in the liver indicating that some phagocytosis had occurred and the particles were delivered to the liver for elimination. Subsequent histology of the tumors also showed significant attachment of the particles to cells in the tumor using Prussian blue staining to emphasize the iron in the magnetic nanoparticles.

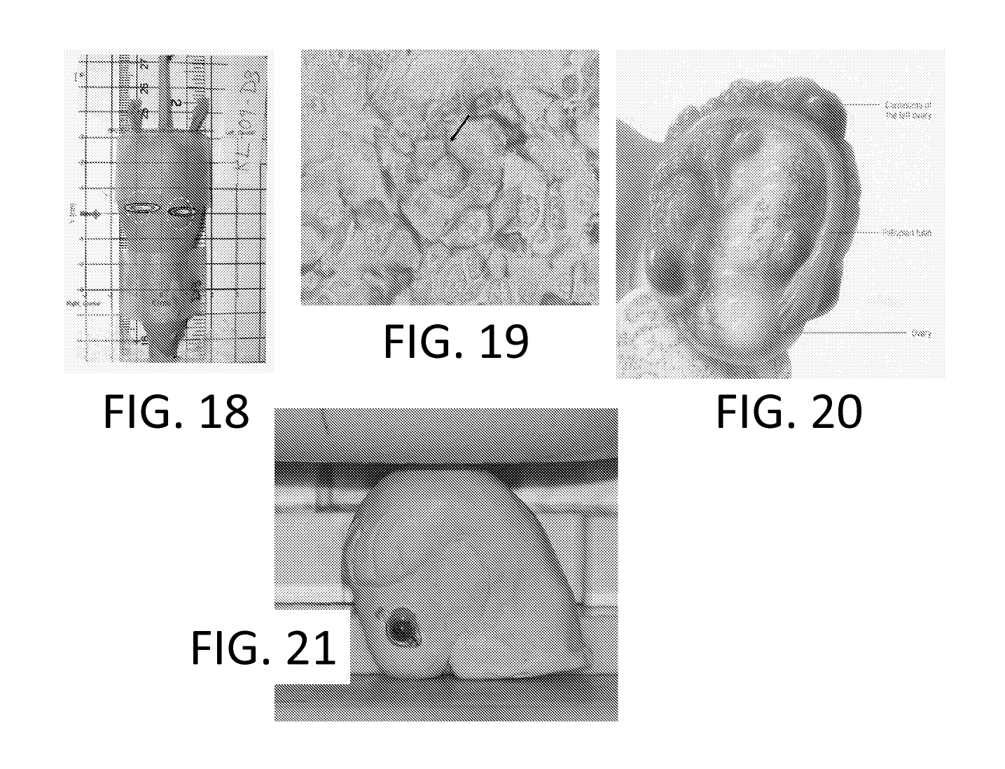

Confidence regions were calculated for determining the accuracy of location of tumors for the in-vivo measurements of the mice. FIG. 18 is an illustration of the 2-dimensional 95% confidence limit for the locations of the two tumors superimposed on the actual tumors of the mouse. An accuracy of spatial location of approximately +1-3 mm is obtained in the x and y direction. FIG. 19 is a photo of the histology of tumors after extraction. Microscopic image of one MCF-7 tumor slice. Prussian Blue staining of cells reveals iron present in np attached to cells. Arrow points to cell covered with np.

A sensitive magnetic field sensor system has been demonstrated for in-vivo early detection of breast cancer by detecting magnetic nanoparticles, conjugated to antibodies for breast cancer cell lines. Hundreds of thousands of nanoparticles attach to each cancer cell. Method is sensitive to <100,000 cells at distances comparable to breast tumors. Standard x-ray mammography requires typically cell density of ten million cells. Measured moments are linear with cell number; i.e. measure of magnetic moment yields the number of cancer cells present. Very high contrast-nanoparticles not attached to cells are not observed. Phantom studies demonstrate multiple sources are localized accurately and number of cells per source determined. Mouse model was developed using multiple tumors of human breast cancer cell lines and in-vivo measurements made to determine the location and cancer cell count of these tumors subsequent to nanoparticle injections. Solutions of the inverse problem successfully locate tumors and number of cells. Histology confirms presence of np mouse tumors.

Example Application to Detection of Ovarian Cancer.

The etiology of ovarian cancer is not well understood and there is little evidence for risk factors suggesting preemptive screening. The normal screening test is pelvic examination if there are suspected symptoms, such as abdominal enlargement, and the results typically reveal advance stage of cancer. Routine screening of women presently is not done as there are no reliable screening tests. The great difficulty now with ovarian cancer is that by the time it is detected, it has metastasized from the ovary into other organs. For this reason, a hysterectomy is often performed along with the ovary removal. If the presence of ovarian cancer can be identified early and is contained in the ovary, the five year survival rate is 95%. However, only 29% are detected at this stage. If the disease has spread locally, this survival rate drops to 72% and if metastasized to distant locations, the rate of survival is 31%. Thus, development of early detection methods is imperative. This example application uses Neel relaxation to provide a measurement specific to ovarian cancer cells; the magnetic signal from Brownian decay of unbound particles in not used in this example.

FIG. 20 is an illustration of an ovarian cancer showing the growth of the tumor on the ovary. These tumors consist of cells with high numbers of receptors for the antibody CA-125 and can be targeted with magnetic nanoparticles labeled with this antibody. FIG. 21 is photograph of a full-size ovarian phantom placed under a SQUID sensor apparatus at a distance that would be typical of a patient subject. The phantom has a vial containing live ovarian cancer cells inserted into it. Magnetic nanoparticles labeled with the antibody CA-125 were inserted into this vial and because these antibodies are highly specific for these ovarian cancer cells, large numbers became attached to the cell surface. These magnetic nanoparticles were then detected by the SQUID sensor apparatus to provide sensitivity calibrations for in-vivo measurements for both animal and human in-vivo models.

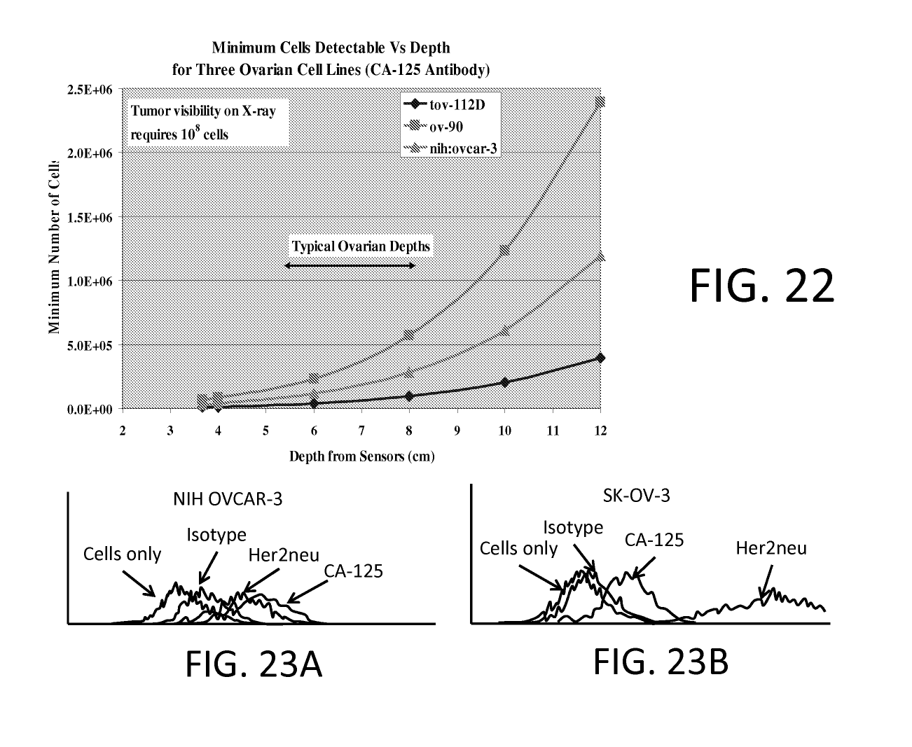

The results of the sensitivity studies for live ovarian cells inserted into the phantom shown in FIG. 21 are illustrated in FIG. 22 for three different ovarian cancer cell lines; namely, tov-112D, Ov-90, and nihovcar-3. The plot shows the minimum number of cells that were detected by this apparatus for the three different cell lines as a function of distance from the sensor to the patient's ovaries. The cancer cell line ov-90 is known to be one of the most aggressive of the cancers and these results indicate that there are many receptors for CA-125 on the surface of the cell. The number of nanoparticles per cell can be estimated from these measurements and corresponds to 20,000 particles per cell for tov-112D, 3400 for ov-90, and 6700 for ovcar-3.

FIG. 23A and FIG. 23B provide an illustration of confirmation of antibody sites for these cells using flow cytometry. FIG. 23A and FIG. 23B show two of the four cell lines examined. The signal from cells only is shown and the isotype (using a non-specific binding molecule, Igg), the Her2/neu antibody, and CA-125 antibody are shown with increasing site number to the right on these plots. These figures show that the CA-125 antibody has a large number of sites on these cells, with SK-OV-3 the largest of these two. The antibody Her2/neu is also specific to 30% of breast cancer cells.

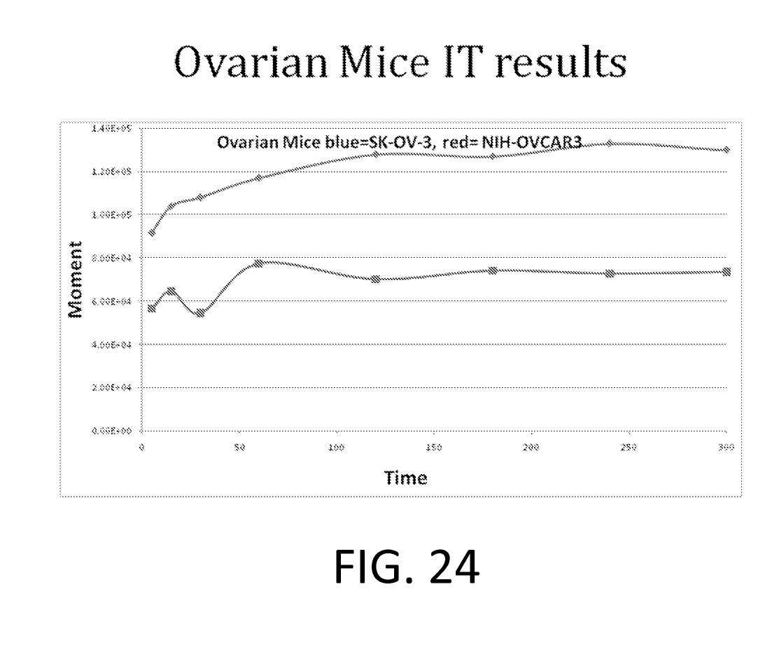

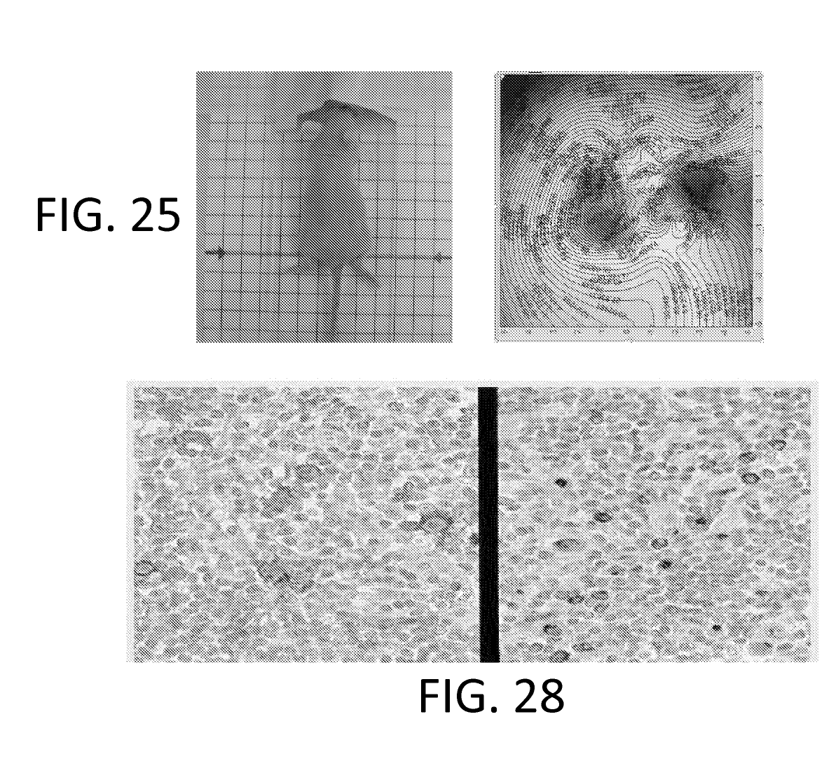

Measurements were made as a function of time to determine how fast the particles were taken up from the blood stream and how fast phagocytosis occurred with the particles ending up in the liver. Measurement of the magnetic moment in the SQUID sensor apparatus as a function of time for magnetic moments from magnetic nanoparticles (from Ocean Nanotech) attached to ovarian human cancer tumors in the live mouse is shown in FIG. 24. The mouse had two ovarian tumors, one of SK-OV-3 and the other of NIH-OVCAR3. The magnetic nanoparticles were coated with a carboxyl biocompatible coating and were then conjugated to the CA-125 antibody. This antibody is specific to ovarian cancer cells in humans. The labeled magnetic nanoparticles were injected into the mouse tumors and the magnetic moments of the mouse measured at various times ranging from one minute to 300 minutes. The uptake of the particles occurred rapidly with the signal near maximum obtained in the first hour. The time course indicates that the nanoparticles remain in the tumors for a number of hours. The nanoparticles remained in the tumors for at least 5 hours, the length of the experiments. Different amounts of nanoparticles were absorbed by each of the two tumors. The mouse tumor with SK-OV-3 cells showed higher magnetic moments than the mouse with NIH-OVCAR-3 tumors, as expected due to the higher number of specific sites for CA-125 antibodies on the former. The nanoparticles gave no magnetic moment before injection and only yield a magnetic signal when attached to something such as the cells in the tumor. Experiments have shown that injections into sites other than the tumor do not yield a signal as the particles do not bind to normal cells. After a period of time, the liver begins to show signs of accumulation of these particles as they are phagocytised from the system. Subsequent to these measurements, the mice were euthanized and the tumors and other organs removed and placed under the sensor apparatus to determine how much of the nanoparticle injections were in the tumors. These measurements agreed very well with the in-vivo measurements on the live mouse. Subsequent histology of the tumors showed significant attachment of the particles to cells in the tumor using Prussian blue staining to emphasize the iron in the magnetic nanoparticles.

A photograph of a mouse used to verify that the SQUID sensor method works in-vivo along with magnetic contour fields from this mouse are shown in FIG. 25. Human xenograft tumors are shown on the flanks of the mouse; these are the bumps above and to both sides of the tail in FIG. 25. These tumors were produced by injecting live human ovarian cancer cells into this severely-compromised-immune-deficient mouse and allowed to grow for several weeks until a 6-10 mm tumor was evident. The mouse was anesthetized through the tube over its mouth during all SQUID sensor experiments. Labeled magnetic nanoparticles were injected into the mouse at this stage either by tail, inter-peritoneal, or inter-tumoral injections. Subsequent to injections, the mouse was placed under a sensor as shown in FIG. 1 and a magnetizing pulse was applied and the resulting magnetic moments of the injected particles was measured. As in the case of the live cancer cells, no moments were observed unless the particles had attached to cells within the tumors. In some cases both tumors were SK-OV-3 type cells and in other cases, two different cell lines were used to develop the tumors in the mice.

The mouse placed on the stage shown in FIG. 5 could be moved to several positions under the sensor to obtain more spatial information. The mouse was typically placed at five stage positions under a 7-channel SQUID to obtain 35 spatial locations. The magnetic fields at all positions were then used in a code to solve the electromagnetic inverse problem using the Levenberg-Marquardt theorem to determine the location of all sources of magnetic particles in the mouse. This information was then compared to the known geometry of the mouse from photographs to determine the accuracy and sensitivity for locating cancer tumors in living animals. FIG. 25 shows the magnetic contour lines observed for 35 different measurements. Analysis of these magnetic fields yielded the spatial positions of the tumors that agreed with the measured values of these positions; the SQUID results giving higher precision than the physical measurements of approximately 3 mm.

Example Application to Detection of Hodgkin's Lymphoma.

Hodgkin's lymphoma (HL) accounts for 30% of all lymphomas. HL characteristically arises in lymph nodes, preferentially in the cervical regions, and thymus; but in advanced disease can involve distant lymph nodes, the spleen, and bone marrow. The majority of cases are in young adults between 15 and 34, but a second incidence peak occurs in people over 55. Currently, biopsy evaluation is required for diagnosis. Surgical biopsy has complications, such as infection and bleeding, and the evaluation of the biopsy typically takes 3-5 days. Thus, in HL cases in which the tumor mass is preventing blood return to the heart (i.e., superior vena cava syndrome, 10% of cases), significant morbidity or mortality can occur during this waiting period. Several of the antibodies that target Hodgkin's lymphoma; namely CD15, CD30, and CD25 have been identified. The latter antibody, however, targets many cells and is less specific. Another application where the present invention can have significant clinical impact is in the detection of persistent HL after therapy. If a patient experiencing a relapse undergoes high-dose radiation therapy, there is a good prognosis if the relapse is detected early. Patients who have a relapse will have a prognosis determined primarily by the duration of the first remission. The persistence of large fibrotic nodules, particularly in the mediastinum, after therapy leads to uncertainty in the determining whether persistent cancer is present and surgery of fibrotic nodules is fraught with difficulty to control bleeding problems and patient morbidity.

The relaxometry method of the present invention can provide a quantitative estimation of the number of lymphoma cells present in organs affected by Hodgkin's disease, such as the thymus and spleen. This example application uses Neel relaxation to provide a measurement specific to cells characteristic of Hodgkin's disease; the magnetic signal from Brownian decay of unbound particles in not used in this example. The RS cells are giant cells derived from B-lymphocytes that contain millions of receptors for CD30 and CD15. Previous results with SQUID sensors targeting T-cell lymphocytes have shown that for smaller cells, approximately a million nanoparticles can be attached to each T-cell. Steric hindrance limits the number of nanoparticles attached to a normal lymphocyte but the much larger RS cells can have 25 to 50 times more bound nanoparticles. The amount of iron per nanoparticle is 4.4×10⁻⁶ ng/np. Given the large size of the RS cells, there can be several million nanoparticles per cell so that each cell may have up to 10 ng of iron. One hundred RS cells accumulated in the spleen or thymus can contain a microgram of iron. Less than a microgram is adequate for SQUID detection, therefore a detectability of 100 RS cells is possible. The measured amplitude of the residual magnetization of the antibody-labeled nanoparticles in vivo can provide an important diagnostic tool in lymphoma cancer. The signal strength depends on the density of antigens on the tumor cell surfaces and thus the field strength produced by the nanoparticles is proportional to the number density of antigenic sites on lymphoma cells. Particle number and density can be determined to provide the amplitude of the detected magnetic field. This information can be used in planning in vivo detection, as well as for assisting in the choice of nanoparticles to be used. The SQUID sensor is an ideal sensor system for Hodgkin's disease with large sensitivity for RS cells and in-vivo detection of the disease without biopsies and the ability to monitor the treatment of the disease during chemotherapy.

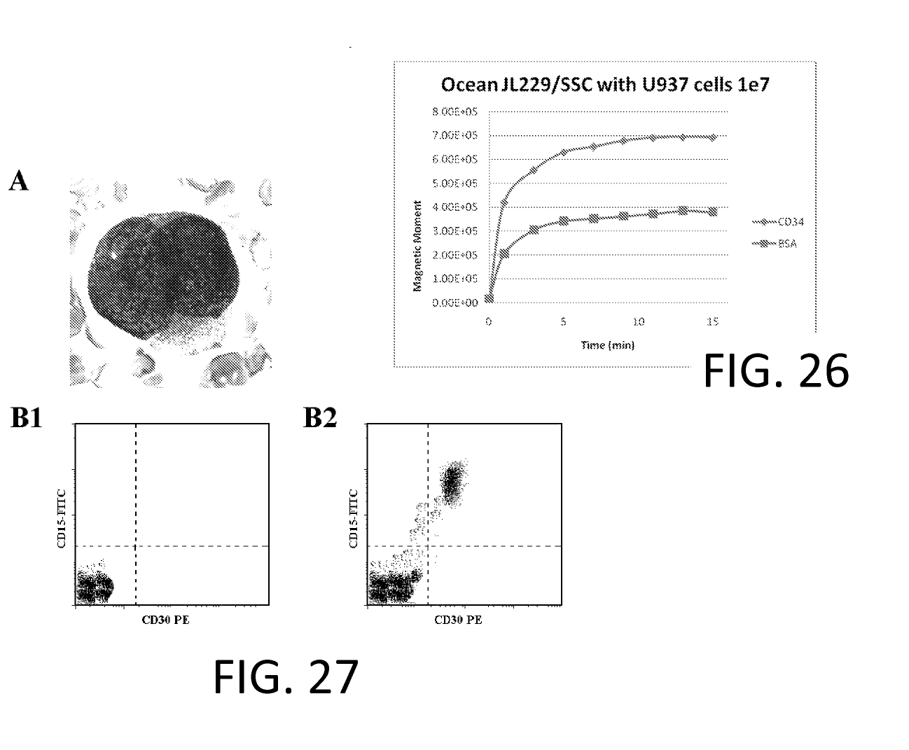

FIG. 26 is a graph of a measurement of the magnetic moment in a SQUID sensor system as a function of time for incubation of attaching magnetic nanoparticles (from Ocean Nanotech) to lymphoma cell lines. The magnetic nanoparticles were coated with a carboxyl biocompatible coating and were then conjugated to the CD34 antibody. This antibody is specific to one type of lymphoma cells, namely, Acute Lymphomatic Leukemia in humans. The labeled magnetic nanoparticles were inserted into vials containing live cancer cells and the magnetic moments of the vial measured at various times ranging from one minute to 16 minutes. The zero time point is the magnetic moment of the vial of nanoparticles before adding to the cells. The lack of magnetic moment for the unmixed particles at time zero shows that unbound particles give no magnetic signal with this SQUID imaging method. Upon mixing with the cells, the magnetic moments increase rapidly and saturate indicating that the cells have collected on their surfaces with the maximum number of nanoparticles possible in one to two minutes. The top curve is for the lymphoma cancer cell line U937 that is known to be very specific for the CD34 antibody and the large magnitude of the magnetic signal verifies this. The lower curve is for the same cell line but a non-specific marker, BSA, and shows substantially smaller magnetic moments after incubation. The presence of a magnetic moment for the BSA is indicative of some phagocytosis of these cells where the nanoparticles enter the cells. U937 is a lymphoma of the T lymphocyte cells and RS is a lymphoma of the B lymphocyte cells. Since one of the principle purposes of lymphocyte cells is to take up particles that do not belong, this amount of non-specificity is expected. These results demonstrate the specificity of the antibody for the target cancer cells and verify that only bound particles give magnetic moments. This result is not true for other methods such as MRI which sees all particles, bound or unbound.

Samples of RS cells were obtained from the Tissue Bank facility at the University of New Mexico, a nationally recognized institution for cell banking and quantity of specimens. The efficiency of the SQUID sensor system for detecting RS cells was compared to the number of RS cells in a sample determined by manual hematocytometer counts. These isolated RS cells were labeled with nanoparticles specificity bound to CD15 and CD30 during the isolation procedure. Calibration of sensitivity was performed by serially dilution over a range of 1 in 10 to 1 in 100,000 cells. Ranges of nanoparticle density on malignant cells exceed 107 nanoparticles/cell. The site density of CD15 is determined using a flow cytometry technique that quantifies receptors/cell. The number of CD15 and CD30 sites/cell was confirmed using a quantitative immunofluorescence staining technique.

FIG. 27 is an illustration of the results of the flow cytometric measurements of RS cells from the lymphatic system in determining the number of sites available for nanoparticles and detection by the SQUID sensors. FIG. 27A is a photograph showing the morphologic appearance of RS cells isolated from a lymph node specimen. FIG. 27B shows the flow cytometric analysis of a bone marrow sample where (B1) is before and (B2) after performing an enrichment procedure to enhance the frequency of RS cells in a sample for flow cytometry. Normally RS cells occur at a frequency of 1 in 10⁴ or 10⁵ of normal lymphocyte cells and must be enhanced before using CD15 and CD30 staining by flow cytometry in order to be detected. The SQUID sensor system detects all of the RS cells in-vivo and does not require sampling so enhancement is not necessary, as is required in the flow cytometry determinations.

The lymph nodes are one of the primary sites where RS cells accumulate, aside from the thymus gland. FIG. 28 is a histology slice from a patient with Hodgkin's Disease. The RS cells have been stained with immunoperoxidase staining. The antibody CD15 is shown on the right and the antibody CD30 on the left. The surrounding cells are non-malignant cells in the lymph node. The SQUID sensor can detect several hundred of these labeled RS cells in a lymph node.

Example Application to Detection and Staging of Prostate Cancer.

Prostate cancer has a high mortality rate due to the lack of early detection with standard screening technologies. The number of cases for 2009 in the US was 192,280 with 27,360 deaths. Prostate cancer accounts for 9% of male deaths and there is a 1 in 6 lifetime probability for developing prostate cancer. The disease is normally undetected until it has caused an enlargement of the prostate, urinary problems, or has spread to other organs. Asymptomatic detection of the disease is normally done by a digital examination, an elevated PSA test result, or a biopsy. The PSA test is now considered unreliable causing many unnecessary biopsies with accompanying dangers of infection. The digital examination is also highly subjective. Testing for prostate cancer is very controversial. The cost of PSA tests in the US alone exceed $3 billion and a recent study reported in the New England Journal of Medicine found that current screening methods do not reduce the death rate in men over 55 years old. The present invention can detect this cancer before it has metastasized. This example application uses Neel relaxation to provide a measurement specific to prostate cancer cells; the magnetic signal from Brownian decay of unbound particles in not used in this example.

An exemplary method to detect prostate cancer in a tissue comprises placing the patient on a measurement stage of a superconducting quantum interference device sensor apparatus; injecting a plurality of antibody-labeled magnetic nanoparticles into the patient for specific binding to the tissue in the patient; applying a uniform magnetizing pulse field to magnetize the nanoparticles injected into the patient; and detecting the residual magnetic field of the magnetized nanoparticles thereby providing an image of the nanoparticles bound to the tissue of the patient. The tissue can comprise prostate tissue and the antibody-labeled magnetic nanoparticles can specifically bind to antigens of prostate cancer cells. The antibody-labeled magnetic nanoparticle can comprise a magnetic core coated with a biocompatible coating to which is attached specific antibodies. For example, the magnetic core can comprise a ferromagnetic material, such as iron oxide. For example, the biocompatible coating can comprise Dextran, carboxyl, or amine. For the detection of prostate cancer, the specific antibody can be PSMA antibody.

The prostate-specific membrane antigen (PSMA) is a transmembrane glycoprotein that is highly expressed by most prostate cancers. It is also referred to as mAb 7E11. It is expressed on the surface of the tumor vascular endothelium of solid carcinomas but not on normal prostate cells. The amount of PSMA observed in prostate cancer follows the severity or grade of the tumor. Flow cytometry has shown that there are large numbers of receptor sites for this antibody on several cell lines of prostate cancer including LNCaP and PC-3, whereas a PSMA negative cell line, DU-145 indicates no expression. Results of attaching magnetic nanoparticles to these positive cell lines demonstrate one million or more nanoparticles per cell. These results are comparable to results from ovarian and breast cancer regarding nanoparticles per cell and depths of tumors in the body, and biomagnetic detection methods using SQUID sensors will have the same sensitivity for prostate cancer as ovarian cancer (described in one or more of the related applications incorporated by reference above). Results of studies on ovarian cancer can thus be directly applied to prostate cancer detection and localization. Compared to the CA-125 antibody for ovarian cancer, the PSMA is even more specific for in vivo prostate specific targeting strategies.

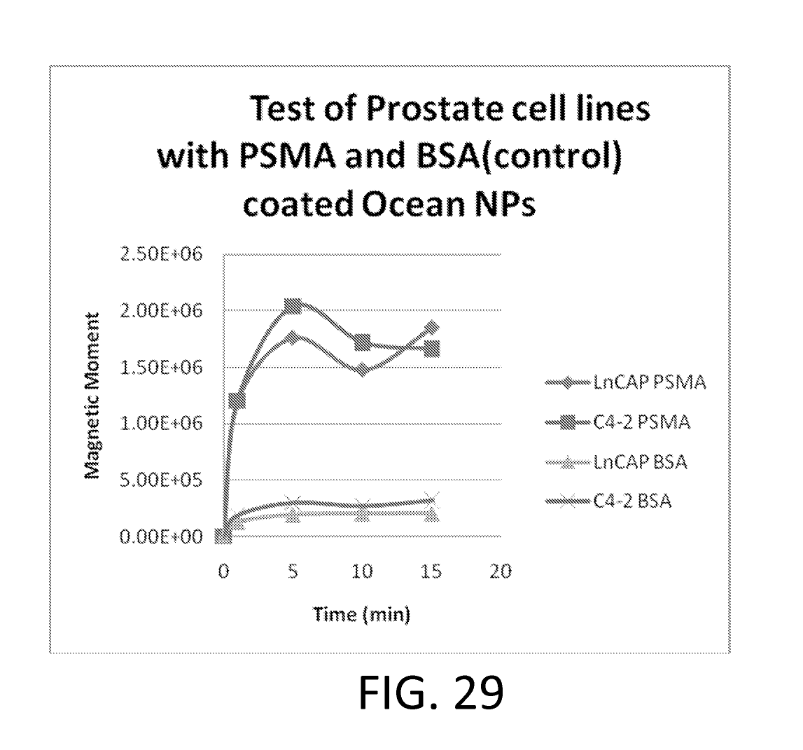

The measurement of prostate cancer cells using an embodiment of the present invention was verified experimentally, as illustrated in FIG. 29. LnCAP and C4-2 are prostate cancer cell lines that are positive for PSMA. 3 million cells of each of these cell lines were exposed to anti-PSMA coated Ocean nanoparticles and BSA coated Ocean nanoparticles. BSA serves as a negative control. As can be seen from the figure, the magnetic moment measured by the SQUID instrumentation is much higher for the PSMA-targeted nanoparticles than for the control nanoparticles.

The SQUID sensor method can provide a quantitative estimation of microvascular structure in tumors leading to a new surrogate for vessel formation (angiogenesis) and individual tumor gradation. It has been shown in a study of tumor microvascular characterization in an experimental prostate cancer model using nanoparticles that tumor growth and aggressiveness/grade have a direct relationship to tumor neovascularization. Other studies estimate the concentration of magnetic particles in a tumor to be about 2.3 mg of nanoparticles per gram of tissue. This concentration is regularly achieved in the tumors of human liver cancer patients receiving treatment via intrahepatic arterially administered radioactive microspheres; the nanoparticles tend to concentrate in the vascular growth ring of a tumor. Less than a nanogram is adequate for SQUID detection. The measured amplitude of the residual magnetization of the antibody-labeled nanoparticles in vivo can provide an important diagnostic tool in prostate cancer. The signal strength depends on the density of antigens on the tumor cell surfaces and thus the field strength produced by the nanoparticles is proportional to the number density of antigenic sites on prostate tumor cells. Thus, particle number and density provides the amplitude of the detected magnetic field. This information can then be used in planning in vivo, as well as for assisting in the choice of nanoparticles to be used.

Example Application to Detection of Glioblastoma.

Brain cancer is particularly deadly and occurs in a number of forms. Cancer involving the glial cells is the most prevalent form and also the most aggressive brain tumor in humans. Various glial cells may be involved causing cancer of the type oligodendroglioma (involving the oligodendrocytes), astrocytoma (involving the astrocytes) and glioblastoma. The latter is the most frequently occurring of the brain cancers. These types of cancer normally results in death within a very short period of time. Gliablastoma cells can be targeted by markers such as EGFR, 8106, and PTN antibodies that may be used to image this type of cancer. Mouse models and brain cancer cell lines, such as U-251, are available for testing before human applications.

An important consideration in targeting brain cancer is the delivery across the blood brain barrier of the nanoparticles with markers attached. This barrier is somewhat opened in the vascular system associated with malignant tumors but still remains an impediment. The use of nanoparticles coated with lipophilic surfaces and then conjugated to antibodies or peptides increases the ability to cross the barrier. Additionally, the nanoparticle with markers can be encapsulated in a polymer coating with a liposome surface of in a micelle is another approach and releasing the conjugated nanoparticles from the polymer once inside of the brain using a slight application of a heating RF or ultrasound pulse. This example application uses Neel relaxation to provide a measurement specific to glioblastoma cells; the magnetic signal from Brownian decay of unbound particles in not used in this example.

An exemplary method to detect brain cancer comprises placing the patient on a measurement stage of a superconducting quantum interference device sensor apparatus; injecting a plurality of antibody-labeled magnetic nanoparticles into the patient for specific binding to the brain tumor in the patient; applying a uniform magnetizing pulse field to magnetize the nanoparticles injected into the patient; and detecting the residual magnetic field of the magnetized nanoparticles thereby providing an image of the nanoparticles bound to the tissue of the patient. The target is a brain tumor and the antibody-labeled magnetic nanoparticles can specifically bind to antigens of brain cancer cells. The antibody-labeled magnetic nanoparticle can comprise a magnetic core coated with a biocompatible coating to which is attached specific antibodies. For example, the magnetic core can comprise a ferromagnetic material, such as iron oxide. For example, the biocompatible coating can comprise Dextran, carboxyl, or amine. For the detection of glioblastomas, the specific antibody can be EGFR or similar antibody.

Angiogenesis EGFR has several forms and is a version of the epidermal growth factor receptor (EGFR) that is overexpressed by several types of cancer cells including glioblastoma cells and not normal cells. EGFR is currently undergoing immunotherapy clinical trials for patients with diagnosed glioblastoma. It can be conjugated with magnetic nanoparticles suitable for magnetic relaxometry detection and injected into the body. These magnetic nanoparticles can comprise a coating, such as polyethylene glycol (PEG), that will increase the efficacy of the targeted nanoparticles for penetrating the blood brain barrier. In another example embodiment of the present invention, the magnetic nanoparticles with markers attached can be contained within polymer coatings that are able to penetrate through the blood brain barrier and then released upon the application of a small RF heating pulse or the use of ultrasound. Results of attaching these angiogenesis peptides to magnetic nanoparticles and attaching these to cells are comparable to the use of other antibody results from ovarian and breast cancer regarding nanoparticles per cell and depths of tumors in the body. Biomagnetic detection methods using systems such as SQUID sensors will have the same sensitivity for brain cancer as ovarian cancer (described in one or more of the related applications incorporated by reference above). Results of studies on breast and ovarian cancer can thus be directly applied to brain cancer detection and localization.

Example Application to Detection of Pancreatic Cancer.

A number of tumor markers are present in pancreatic cancer. CA19-9 is one example of a marker that is elevated in this cancer but is not very sensitive (77%) and non-specific (87%). Combinations of markers have been suggested by the M.D. Anderson Cancer Center and these are being tested for screening of pancreatic cancer. These markers are microRNAs and include miR-21, MiR-210, miR-155 and miR-196a. However, this combination also only achieves a low sensitivity (64%) but a higher specificity (89%) than the CA19-9. In addition, a number of antibodies have been identified against certain cell lines of human pancreatic cancer, for example the FG cell line and these include S3-15, S3-23, S3-41, S3-60, S3-110, and S3-53. Another marker is muclpan4 that is shown to be expressed in over 90% of pancreatic cancers. Another identifying marker is the urokinase plasminogen activator receptor (uPAR) that is highly expressed in pancreatic cancer and also in tumor stromal cells. The latter marker has been used to deliver magnetic nanoparticles to pancreatic cancers grown as xenografts in nude mice. These markers have led to MRI detection of the tumors in the mice when used as labeled contrast agents. The mechanism is primarily delivery of the nanoparticles to the tumor endothelial cells. This example application uses Neel relaxation to provide a measurement specific to pancreatic cancer cells; the magnetic signal from Brownian decay of unbound particles in not used in this example.

There are no reliable imaging approaches for diagnosis of pancreatic cancer. Thus the development of biomarkers as a targeted imaging agent for MRI, or permitting the more sensitive technique of magnetic relaxometry, is a significant advance. MRI can detect small abnormalities in tumors and is also useful in determining if cancer has metastasized. Dynamic Contrast Enhanced (DCE) MRI potentially distinguishes between benign and cancerous tumors but produces a number of false positives. The expense of MRI limits its application as a screening tool. MRI imaging of tumors often uses magnetic nanoparticles as contrast agents as mentioned above and is an accepted protocol providing standards for the injection of such nanoparticles. Intravascular MRI contrast agents at a dose of 2 mg/kg of nanoparticle weight have been used to detect metastatic lesions. However, the use of MRI in pancreatic cancer is severely limited.

The present invention can provide a quantitative estimation of microvascular structure in tumors leading to a new surrogate for vessel formation (angiogenesis) and individual tumor gradation. It has been shown in results in a study of tumor microvascular characterization in an experimental pancreatic cancer model using nanoparticles that tumor growth and aggressiveness/grade have a direct relationship to tumor neovascularization. Other studies estimate the concentration of magnetic particles in a tumor of ˜2.3 mg of nanoparticles per gram of tissue. This concentration is regularly achieved in the tumors of human liver cancer patients receiving treatment via intrahepatic arterially administered radioactive microspheres; the nanoparticles tend to concentrate in the vascular growth ring of a tumor. Nanograms are adequate for detection by the present invention. The measured amplitude of the residual magnetization of the antibody-labeled nanoparticles in vivo can provide an important diagnostic tool in pancreatic cancer. The signal strength depends on the density of antigens on the tumor cell surfaces and thus the field strength produced by the nanoparticles is proportional to the number density of antigenic sites on pancreatic tumor cells. Particle number and density can be determined to provide the amplitude of the detected magnetic field. This information can be used in planning in vivo detection, as well as for assisting in the choice of nanoparticles to be used. Examples of pancreatic cancer cell lines include FG or MIA PaCa-2 that are known to be specific for the uPAR antibody.

Example Application to Detection of Sentinel Nodes.

FIG. 31 is a schematic illustration of the application of the present invention to detection of sentinel nodes. At the top left of the figure, nanoparticles are introduced into a first region of the patient. The nanoparticles can be superparamagnetic nanoparticles, wherein the nanoparticles, when in a substance having a viscosity like that the viscosity in the second region, undergo Brownian motion that randomizes the orientation of the nanoparticles according to a predetermined characteristic time. As examples, nanoparticles can be selected as described in connection with FIG. 30.

At the top right of the figure, the nanoparticles have been transported to a second region of the body. As an example, if the nanoparticles were introduced into a tumor site, then the illustration at the top right can correspond to drainage from the tumor site to a sentinel lymph node.

At the lower left of the figure, the second region of the body is subjected to an applied magnetic field. The applied magnetic field can be of sufficient strength to induce magnetization of individual nanoparticles, and can have a substantially uniform direction throughout the second region. As an example, an applied field of about 50 Gauss, for less than 10 seconds, or less than 1 second, can be suitable with nanoparticles like those described above. The applied magnetic field magnetizes the nanoparticles present in the second region, and aligns the magnetic moments of the magnetized nanoparticles.

At the lower right of the figure, the applied magnetic field is no longer being applied. The magnetic field in the second region can be measured, for example using methods and apparatuses like those described above. The measured magnetic field will be affected by the remaining magnetization of the nanoparticles in the second region. The net magnetic field in the second region will decay as the moments of the nanoparticles attain random orientations due to Brownian motion of the nanoparticles, as depicted schematically to the right of the patient outline in the figure. The rate of randomization can be determined from the known characteristics of the nanoparticles, and a component of the measured magnetic field that corresponds to the expected decay rate can be determined. The magnitude of that signal corresponds to the number of nanoparticles that have reached the second region, and thus the transport of nanoparticles from the first region to the second region can be determined. In some embodiments, the magnetic field can be measured at a plurality of points, and the spatial location or distribution of the nanoparticles also determined, as described above. For example, solving the electromagnetic inverse problem using the detected signals at a plurality of points can allow determination of the location of the lymph node to which the nanoparticles drained.

The present invention has been described as set forth herein in relation to various example embodiments and design considerations. It will be understood that the above description is merely illustrative of the applications of the principles of the present invention, the scope of which is to be determined by the claims viewed in light of the specification. Other variants and modifications of the invention will be apparent to those of skill in the art.

What is claimed is:

1. A method of determining the communication of substances between a first region and a second region of a patient's body, comprising:
(a) introducing into the first region a plurality of superparamagnetic nanoparticles, wherein the nanoparticles, when in a substance having a viscosity like that the viscosity in the second region, undergo Brownian motion that randomizes the orientation of the nanoparticles according to a predetermined characteristic time;
(b) after a time sufficient to allow transport of nanoparticles from the first region to the second region, subjecting the second region to an applied magnetic field of sufficient strength to induce magnetization of individual nanoparticles, and having a substantially uniform direction throughout the second region;
(c) measuring the magnetic field of the second region at a plurality of times after ceasing application of the magnetic field;
(d) analyzing the measured magnetic field to detect signals that correspond to decay of the magnetic field due to randomization of the nanoparticles' orientation by Brownian motion;
(e) determining the presence of nanoparticles in the second region from the signals detected in step (d).

2. A method as in claim 1, wherein the characteristic time is such that a magnetic field from magnetized nanoparticles decays to one half its original strength in less than 10 seconds.

3. A method as in claim 2, wherein the characteristic time is such that a magnetic field from magnetized nanoparticles decays to one half its original strength in less than 1 second.

4. A method as in claim 1, wherein step (c) comprises measuring the magnetic field at a plurality of locations, and wherein step (d) comprises determining locations in the second region wherein the measured magnetic field indicates the presence of nanoparticles.

5. A method as in claim 1, wherein the magnetic field is measured in step (c) at a plurality of times after ceasing application of the magnetic field.

6. A method as in claim 1, wherein the magnetic field in step (b) has a strength of about 50 Gauss.

7. A method as in claim 1, wherein the magnetic field in step (b) is applied for less than ten seconds.

8. A method as in claim 7, wherein the magnetic field in step (b) is applied for less than one second.

9. A method as in claim 1, wherein measuring the magnetic field in step (c) comprises using one or more superconducting quantum interference devices to measure the magnetic field.

10. A method as in claim 1, wherein measuring the magnetic field in step (c) comprises using one or more atomic magnetometers to measure the magnetic field.

11. A method as in claim 1, wherein measuring the magnetic field in step (c) comprises using one or more magnetic sensors coupled to one or more second order gradiometers to measure the magnetic field.

12. A method as in claim 1, wherein measuring the magnetic field in step (c) comprises using a plurality of magnetic sensors to measure the magnetic field, including measuring spatial characteristics of the magnetic field.

13. A method as in claim 12, wherein step (d) comprises determining a spatial distribution of the nanoparticles.

14. A method as in claim 12, wherein step (d) comprises solving an inverse electromagnetic problem to determine locations of magnetic sources in the sample.

15. A method as in claim 1, further comprising repeating steps (b) through (d) a plurality of times and averaging the magnetic field measurement in step (c), the particle determination in step (e), or a combination thereof, of two or more of such repetitions of steps (b) through (e).

16. A method as in claim 1, wherein step (d) comprises identifying a component of the magnetic field that fits a decay curve comprising a log/exponential function.

17. An apparatus for determining the communication of substances between a first region and a second region of a patient's body, comprising:
(a) a magnetization system, configured to subject a sample to a magnetic field, wherein the sample has been exposed to a plurality of superparamagnetic nanoparticles; wherein the magnetic field has sufficient strength to induce magnetization of individual nanoparticles;
(b) a magnetic measurement system, configured to measure a magnetic field of the sample at a plurality of measurement times after a magnetic field applied by the magnetization system has been decreased below a threshold;
(c) an analysis system, configured to analyze the measured magnetic field to detect signals that correspond to decay of the magnetic field due to randomization of the nanoparticles' orientation by Brownian motion, and to determine the presence of nanoparticles in the second region from the signals detected.

18. An apparatus as in claim 17, wherein the magnetic measurement system comprises one or more superconducting quantum interference devices.

19. An apparatus as in claim 17, wherein the magnetic measurement system comprises one or more atomic magnetometers.

20. An apparatus as in claim 17, wherein the magnetic measurement system comprises one or more magnetic sensors coupled to one or more second order gradiometers.

21. An apparatus as in claim 17, wherein the magnetic measurement system comprises a plurality of magnetic sensors configured to measure spatial characteristics of the magnetic field, and wherein the analysis system is configured to determine spatial distribution of the nanoparticles from the spatial characteristics of the magnetic field.
